(19) United States
(12) Patent Application Publication
(10) Pub. No.: US 2012/0010167 A1
(43) Pub. Date: Jan. 12, 2012

(54) COUNTERMEASURE METHODS AND DEVICES

(76) Inventor: Kenton W. Gregory, Portland, OR (US)

(21) Appl. No.: 13/257,040

(22) PCT Filed: Mar. 19, 2010
(86) PCT No.: PCT/US10/27984

§ 371 Date: Sep. 16, 2011

Related U.S. Application Data
(60) Provisional application No. 61/161,500, filed on Mar. 19, 2009.

(51) Int. Cl. A61K 31/722; A61Q 17/00; A61P 39/04; A61P 31/12; A61P 31/04

(52) U.S. Cl. 514/55

(57) ABSTRACT

Embodiments of the present invention provide methods for protecting against toxic agents using chitosan formulations.

### TECHNICAL FIELD

Embodiments of the present invention relate to methods and devices for protecting against toxic agents.

### BACKGROUND

Weaponized agents, including chemical warfare agents, biological or biologic warfare agents, nuclear agents and radiological agents, remain a potentially devastating threat to soldiers and civilians. Exposure to such toxic agents can markedly reduce military force effectiveness and can consume limited resources in the field.

Soldiers, government civilian employees and civilians can be exposed to ionizing radiation from gases, liquids and microparticulates containing radioactive isotopes. Life threatening exposures to other toxic metals and chemical agents occur in a variety of military and civilian settings. Prevention, minimization and treatment of exposures are of paramount importance for force protection and civilian safety. Current protection and treatment measures are mostly agent specific and thus have an extremely narrow range of effectiveness with respect to the very broad array of radiation, chemical or microbial agents.

Depleted uranium, or U-238 (“DU”), is a toxic, heavy metal byproduct of uranium enrichment that gives the world uranium suitable for use in nuclear weapons and reactor fuel. It is also used in munitions, ballast for airplanes, tank armor and products. Researchers found that even though the alpha radiation from depleted uranium is relatively low, internalized DU as a metal can induce DNA damage and carcinogenic lesions in the cells that make up bones in the human body. The material in armor-piercing munitions ignites and burns on impact at temperatures of several thousand degrees Celsius. Burning particles or dust of uranium oxide aerosol are created and may be carried considerable distances by winds.

A safe, inexpensive, and simple foundation technology capable of extremely broad protection and treatment against many threat agents is needed. This requires a technology that manifests highly diverse binding capabilities.

### SUMMARY

It has been found that chitosan is useful for adsorbing and/or binding to heavy metals, radioisotopes and other toxic agents.

In accordance with embodiments of the present invention, chitosan and derivatives thereof may be included or suspended in lotions, gels or skin creams that are applied to skin. In another embodiment, a suspension of chitosan in a cosmetically acceptable carrier is packaged in a portable container, e.g., a tube or bottle, for use on the skin to periodically inactivate toxic agents held on a person's skin.

In still another embodiment, the chitosan hereof may be ingested for internal interaction and inactivation of toxic agents within the gastrointestinal tissue. When wastes are expelled, the toxic agents are retained on the chitosan and prevented from causing damage or harm.

### DETAILED DESCRIPTION OF EMBODIMENTS OF THE INVENTION

In the following detailed description, reference is made to embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete steps in turn, in a manner that may be helpful in understanding embodiments of the present invention; however, the order of description should not be construed to imply that these operations are order dependent.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

In various embodiments of the invention, methods and devices for inactivating toxic agents is provided. In the following description, unless further particularized or otherwise noted, the term “toxic agents” or “toxic agent” is intended to refer to a broad class of poisonous substances, including but not limited to toxic gases and/or particulates, radioactive gases and/or particulates, weaponized agents, pathogens, and/or microbes, The term “pathogen” or “pathogens” is intended to refer to any bacterium, virus or other microorganism that can cause disease. The term “inactivate” or “inactivating” is intended to refer to rendering a toxic agent ineffective at causing disease or harmless to animals or humans.

Although certain embodiments have been described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

Chitosan, a chemical derivative of chitin (the second most abundant material in the earth's biomass), appears to fulfill the need for broad-based binding abilities while satisfying requirements for safety and applicability. Chitosan is a partially N-deacetylated derivative of chitin, a polysaccharride found in the shell of crustaceans such as shrimp, crabs and lobsters, insects or mollusks. It is consists of 2-acetamido-2-deoxy-D-glucopyranose units linked together by 1,4-glycosidic bonds. Chitosan has an excellent selectivity and affinity to heavy metal anions and cations. It is generally assumed that the cationic nature of chitosan (pKa 6.3), conveyed by the positively charged NH3 groups of glucosamine, might be a fundamental factor contributing to its interaction with heavy metal ions. These amine groups interact with the metal ions through chelating or electrostatic attraction, depending on pH and the nature of the metal ions and may coordinate with other groups and molecules which may explain the useful capacity to bind heavy metals of differing valences. At pH values close to neutrality or in the non-protonated form, the free electron doublet on nitrogen of amine group may be used to form donor bonds with coordination transition metals such as Cu, Ni, Hg, Zn, etc. At low pH, where protonation of the amine group takes place, the cationic groups of chitosan may bind anions through electrostatic interactions.

The term “chitosan,” as described herein, generally refers to a deacetylated derivative of chitin. The term “chitosan” may include both ideal chitosan, which is a linear polysaccharide of 2-acetamido-2-deoxy-D-glucopyranose, as well as any chitin/chitosan co-polymer such as linear polysaccharides composed of randomly distributed β-(1-4)-linked D-glucosamine (deacetylated unit) and N-acetyl-D-glucosamine (acetylated unit).

Chemical modification or derivatization of chitosan may be useful for modifying or enhancing the bioactivity of native chitosan. Accordingly, various embodiments of the present invention may comprise chemically modified chitosan or chitosan derivatives.

Chitosan preparations are potentially capable of binding a myriad of toxic agents whether chemical, radioactive or microbial. Chitosans and chitosan salts have a remarkable binding affinity for bacteria, viruses, endotoxins, proteins, heavy metals and other chemical agents. Chitosan's safety profile for humans and animals has long been demonstrated. It has been used in Asian cultures as a wound healing material for generations, is a primary ingredient in FDA approved medical devices (most recently as a hemorrhage control dressing deployed for US troops), and is FDA GRAS approved as a food additive. Chitosans have been used in large quantities as a water purification flocculent and have been used to combat viral and fungal infections in plants.

Chitosan is a known effective chelator of heavy metal ions and thus may be an ideal candidate for binding uranium and other heavy metal isotopes of great threat potential.

Skin Protection

The invention provides a methods for protecting against and/or removing toxic agents from human or animal skin such methods comprising applying a topical formulation to a portion of skin, wherein the topical formulation comprises chitosan or a derivative thereof and a cosmetically acceptable carrier.

Skin compositions based on chitosan may be applied with minimal or no skin irritation for long periods. These compositions may covalently bind or chelate the salts of heavy metal radioactive isotopes that exist in solution, gas or as aerosolized microparticles. Chitosan is not absorbed through the skin and may prevent adsorption of these agents into tissues.

In one embodiment, a suspension of chitosan in a cosmetically acceptable carrier is packaged in a portable container, e.g., a tube or bottle, for use on the hands to periodically inactivate toxic agents held on a person's skin. For example, chitosan compositions may form a protective hydrogel that can be washed off with water. These compositions could be routinely applied prior to anticipated exposure. Alternatively, they could be used to bind and remove toxic agents from previously exposed skin or wounds. Chitosan's diverse binding characteristics may be ideal for protecting against a broad range of heavy metal isotopes.

Cosmetic formulation” entails an active component and the excipients (typically dermatologically-acceptable) employed in cosmetics. The term “dermatologically-acceptable,” as used herein, means that the compositions or excipient components thereof are suitable for use in contact with human skin without undue toxicity, incompatibility, instability, allergic response, or the like.

The compositions according to the invention also comprise a liquid, solid or semi-solid physiologically and cosmetically acceptable vehicle or carrier. A suitable vehicle, under the invention, may act variously as solvent, diluent or dispersant for the constituents of the composition, and allows for the uniform application of the constituents to the surface of the skin at an appropriate dilution. It will be apparent to the skilled artisan that the range of possible vehicles is very broad. In general, compositions according to this invention may comprise water as a vehicle, and/or at least one physiologically and cosmetically acceptable vehicle other than water.

When water comprises a vehicle in compositions under the invention, preferably the water is deionized. Vehicles other than water that can be used in compositions under the invention may be liquids or solids, including emollients, various solvents, powders, and humectants. Carriers, including water, may be used singly or in combination. Suitable carriers may include, but are not limited to, the following examples: water castor oil, ethylene glycol monobutyl ether, diethylene glycol monoethyl ether, corn oil, dimethyl sulfoxide, ethylene glycol, isopropanol, soybean oil, glycerin, soluble collagen, zinc oxide, titanium oxide, talc, Kaolin, hyaluronic acid.

The active constituents of the skin care compositions according to the invention may be soluble or insoluble in a liquid carrier. If the active constituents are soluble in the carrier, the carrier acts as solvent for the active ingredient. If the active constituents are insoluble in the carrier, they are dispersed in the carrier by means of, for example, a suspension, emulsion, gel, cream or paste, and the like. A preferred vehicle to act as solvent and/or diluent for the active constituents is water. Various oils, such as vegetable oils obtained from any of corn, sunflower, safflower, soybean, canola, and the like, may also be used as a vehicle, either alone or in combination. Various oils may also be used in combination with water following emulsification.

The composition may optionally comprise other cosmetic actives and excipients, obvious to those skilled in the art including, but not limited to, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, depigmenting agents, hypopigmenting agents, preservatives (e.g., DMDM Hydantoin/lodopropynylbutylcarbonate), stabilizers, pharmaceutical agents, photostabilizing agents, neutralizers (e.g., triethanolamine) and mixtures thereof. In addition to the foregoing, the cosmetic compositions of the invention may contain any other compound for the treatment of skin disorders.

The compositions according to the invention may also comprise an acid including, but not limited to, acetic acid and citric acid. The concentration of the acid may be adjusted slightly to provide a suitable pH. In the case of the skin composition, the preferred pH is in the range of 6.0 to 8.0; more preferably the pH is in the range of 6.5 to 7.5.

In accordance with embodiments of the present invention, chitosan and/or derivatives thereof may be included or suspended in a topical formulation for protecting against and/or removing toxic agents from human or animal skin. For such embodiments, the topical formulation may be a cream, lotion, spray or gel.

In various embodiments of the present invention, the toxic agents targeted by the topical formulation may include one or more pathogens. For example the toxic agents may include one or more viruses or bacteria. For such embodiments, the topical formulation may be capable of substantially inactivating the pathogens.

In accordance with various embodiments, the topical chitosan formulation may be capable of substantially sequestering toxic agents. In other embodiments, the topical chitosan formula may be capable of substantially chelating one or more heavy metals, or radioactive agents.

In accordance with various embodiments, the topical chitosan formulation may be applied to a portion of human or animal skin to protect against and/or remove toxic agents the portion of skin. In various embodiment, the topical chitosan formulation may be applied prior to an exposure to the toxic agents. In other embodiments, the topical chitosan formulation may be applied after to an exposure to the toxic agents. In additional embodiments, the topical formulation may be removed to remove the toxic agents from the skin.

Prevention and Treatment Gastrointestinal Exposures

The invention further provides methods for removing toxic agents from human or animal gastrointestinal tissue, such methods comprising ingesting a therapeutic formulation, wherein the therapeutic formulation comprises chitosan or a derivative thereof and a physiologically acceptable carrier.

In accordance with this embodiment of the present invention, chitosan preparations could be given to bind radioactive isotopes in the gut to prevent or reduce adsorption of these agents locally and systemically, and then usher these agents quickly out of the GI tract via a variety of common methods. For such embodiments, the therapeutic formulation may be in the form of a liquid, pill or lozenge.

In accordance with various embodiments, the therapeutic chitosan formulation may be capable of substantially sequestering toxic agents. In other embodiments, the therapeutic chitosan formula may be capable of substantially chelating one or more heavy metals, or radioactive agents.

The term “physiologically acceptable” generally refers to a material that does not interfere with the effectiveness of an IRO compound and that is compatible with a biological system such as a cell, cell culture, tissue, or organism. Preferably, the biological system is a living organism, such as a vertebrate.

The term “carrier” generally encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

In additional embodiments of the present invention, a therapeutic chitosan formulation may be administered via a catheter or enema.

What is claimed is:

1. A method for protecting against and/or removing toxic agents from human or animal skin comprising applying a topical formulation to a portion of skin, wherein the topical formulation comprises chitosan or a derivative thereof.

2. The method of claim 1, wherein the one or more toxic agents include one or more pathogens, heavy metals, or radioactive agents.

3. The method of claim 2, wherein the one or more pathogens include one or more viruses or bacteria.

4. The method of claim 1, wherein the topical formulation is a cream, lotion or gel.

5. The method of claim 1, wherein the chitosan or a derivative thereof is capable of substantially sequestering the toxic agents.

6. The method of claim 1, wherein the chitosan or a derivative thereof is capable of substantially chelating the toxic agents.

7. The method of claim 1, wherein the chitosan or a derivative thereof is capable of substantially inactivating the toxic agents.

8. The method of claim 1, wherein said applying a topical formulation to a portion of skin occurs prior to an exposure to the toxic agents.

9. The method of claim 1, wherein said applying a topical formulation to a portion of skin occurs after to an exposure to the toxic agents.

10. The method of claim 1, further comprising removing the topical formulation from the portion of skin following an exposure to the toxic agents.

11. A method for removing toxic agents from human or animal gastrointestinal tissue comprising ingesting a therapeutic formulation, wherein the therapeutic formulation comprises chitosan or a derivative thereof and a physiologically acceptable carrier.

12. The method of claim 11, wherein the one or more toxic agents include one or more heavy metals, or radioactive agents.

13. The method of claim 11, wherein the therapeutic formulation is a liquid, pill or lozenge.

14. The method of claim 11, wherein the chitosan or a derivative thereof is capable of substantially sequestering the toxic agents.

15. The method of claim 11, wherein the chitosan or a derivative thereof is capable of substantially chelating the toxic agents.

16. The method of claim 11, wherein the chitosan or a derivative thereof is capable of substantially inactivating the toxic agents.
